(19) United States
(12) Patent Application Publication
(10) Pub. No.: US 2007/0225295 A1
(43) Pub. Date: Sep. 27, 2007

(54) Ziprasidone Hydrochloride Polymorph and Process for Its Preparation

(75) Inventors: Gianpiero Ventimiglia, Cinisello Balsamo (IT); Pietro Allegrini, San Donato Milanese (IT); Graziano Castaldi, Briona (IT)

Correspondence Address: GIFFORD, KRASS, SPRINKLE,ANDERSON & CITKOWSKI, P.C, PO BOX 7021, TROY, MI, 48007-7021, US

(73) Assignees: DIPHARMA S.P.A., Mereto Di Tomba (IT); LUNDBECK PHARMACEUTICALS ITALY S.P.A., Padova (IT)

(21) Appl. No.: 11/568,913

(22) PCT Filed: May 10, 2005
(86) PCT No.: PCT/EP05/52091

§ 371 Date: Nov. 10, 2006

(30) Foreign Application Priority Data
May 11, 2004 (IT) MI2004A000944

(51) Int. Cl. A61K 31/496; C07D 417/12

(52) U.S. Cl. 514/254.04; 544/368

(57) ABSTRACT

New crystalline form of ziprasidone hydrochloride hemihydrate, process for its preparation, its use for the purification of ziprasidone, its pharmaceutical compositions and their use in therapy.

### FIELD OF THE INVENTION

The present invention relates to a new polymorphic form of ziprasidone hydrochloride, in particular its approximately hemihydrate crystalline form, a process for its preparation, its pharmaceutical composition and its use in therapy. The invention also provides a method for purifying a crystalline form of ziprasidone hydrochloride monohydrate, hemihydrate or anhydrate using said new approximately hemihydrate crystalline form.

### PRIOR ART

Ziprasidone hydrochloride, 5-[2-[4-(1,2-benzisothiazol-3-yl)-1-piperazinyl]ethyl]-6-chloro-1,3-dihydro-2H-indol-2-one hydrochloride, having the following formula, 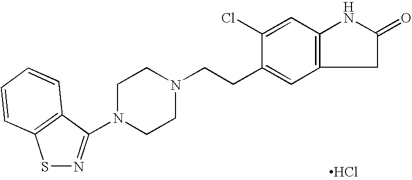
is known from U.S. Pat. No. 4,831,031 and is used in medicine as an antipsychotic drug. Ziprasidone hydrochloride, as referred to in U.S. Pat. No. 5,312,925, is known to exist in three different crystalline forms: the first is a monohydrate form having a water content equal to 3.97% by weight and characterised by the X-ray powder diffraction spectrum, XRPD, shown in FIG. 1 of the accompanying drawing; the second is a hemihydrate form, characterised by a water content equal to 2.55% by weight and by the X-ray powder diffraction spectrum shown in FIG. 2 of the accompanying drawings; the third is a substantially anhydrous crystalline form, indicated here as “crystalline anhydrate” for brevity, with a water content equal to 0.13% by weight characterised by the X-ray powder diffraction spectrum shown in FIG. 3 of the accompanying drawings.

### SUMMARY OF THE INVENTION

In accordance with the present invention, it has now been found that ziprasidone hydrochloride can also exist, in addition to said known crystalline monohydrate, hemihydrate and anhydrate forms, in a new approximately hemihydrate polymorphic form being stable at ambient temperature. A first aspect of the invention is therefore a new approximately hemihydrate crystalline form of ziprasidone hydrochloride, characterised by having a XRPD spectrum wherein the most intense diffraction peaks are found at 7.44; 12.60; 13.89; 17.88; 20.94 and 25.98 in 2θ.

An aspect of the invention described herein is also a process for preparing said approximately hemihydrate form of ziprasidone hydrochloride.

A further aspect of the invention is the use of the new approximately hemihydrate crystalline form of ziprasidone hydrochloride as intermediate in a method for purifying ziprasidone hydrochloride to obtain crystalline ziprasidone hydrochloride monohydrate, hemihydrate and anhydrate of a quality suitable for therapeutic use. As a final aspect the invention also provides a pharmaceutical composition comprising at least one diluent and/or carrier and, as active principle, said new approximately hemihydrate crystalline form of ziprasidone hydrochloride and optionally at least one of the other known crystalline forms, and its use in therapy. X-ray powder diffraction, XRPD, was also used to characterise the new crystalline form of the invention. Moreover, the water content of the compound under examination was determined by titration with the known Karl-Fischer method and the chloride content of the product was titrated potentiometrically. The X-ray diffraction spectra (XRPD) were gathered with a θ/θ automated powder and liquid diffractometer, an APD-2000 from the ltal-Structures company, under the following operative conditions: CuKα radiation (λ=1.5418 Å), scanning with angular step size of 0.03° for a 1 second period.

### BRIEF DESCRIPTION OF FIGURES

The figures of the accompanying illustrations show:

FIG. 1. XRPD spectrum of ziprasidone hydrochloride monohydrate known from U.S. Pat. No. 5,312,925.

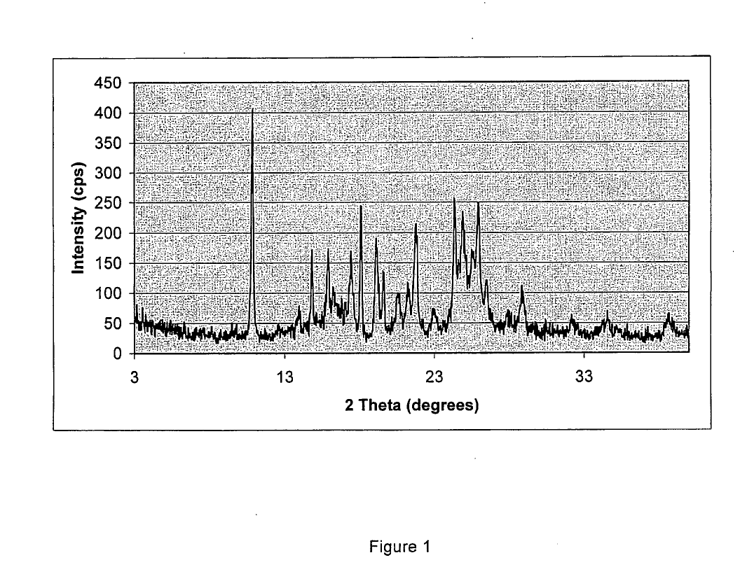

FIG. 2. XRPD spectrum of ziprasidone hydrochloride hemihydrate known from U.S. Pat. No. 5,312,925 wherein the most intense diffraction peaks are found at 11.28; 18.09; 19.47; 23.67 and 26.16 in 2θ.

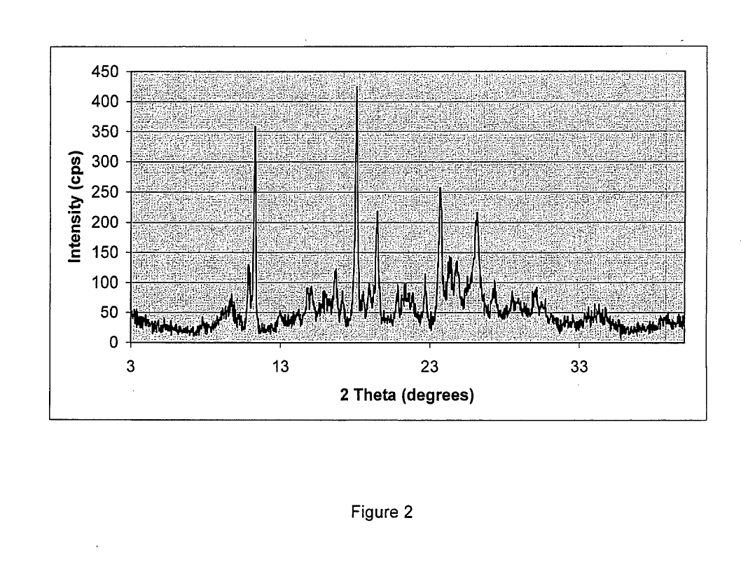

FIG. 3. XRPD spectrum of ziprasidone hydrochloride anhydrate known from U.S Pat. No. 5,312,925.

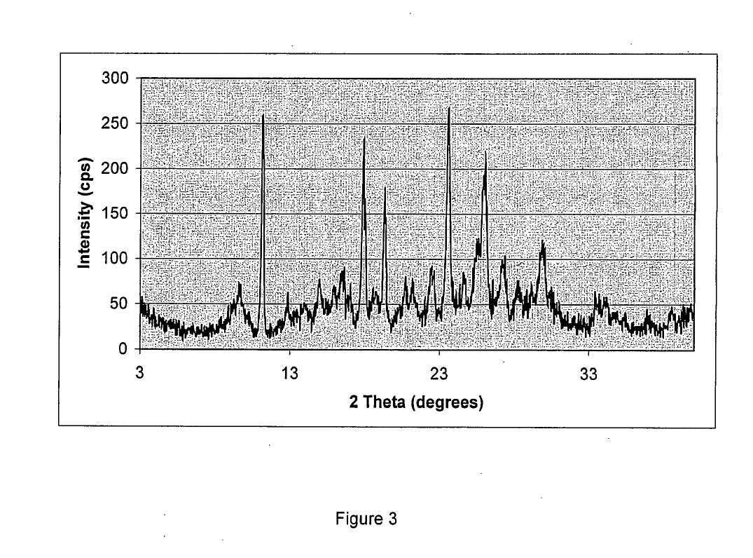

FIG. 4. XRPD spectrum of the new polymorphic approximately hemihydrate form of ziprasidone hydrochloride of the invention wherein the most intense diffraction peaks are found at 7.44; 12.60; 13.89; 17.88; 20.94 and 25.98 in 2θ.

### DETAILED DESCRIPTION OF THE INVENTION

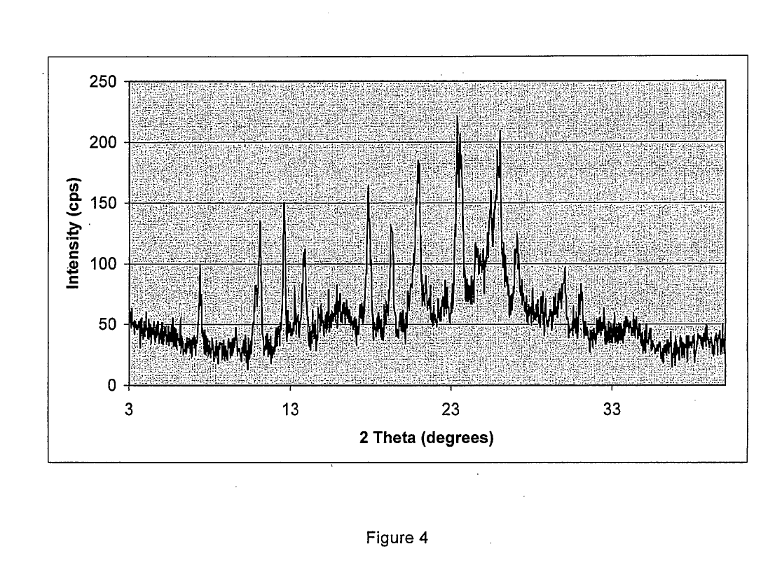

Therefore, a first aspect of the present invention is the new approximately hemihydrate crystalline form of ziprasidone hydrochloride, having a XRPD spectrum as shown in said in FIG. 4, wherein the most intense diffraction peaks are found at 7.44; 12.60; 13.89; 17.88; 20.94 and 25.98 in 2θ.

In accordance with the present invention, the term “approximately hemihydrate” means that the crystalline solid has a water content of about 0.4 to 0.6 moles per mole of ziprasidone hydrochloride, preferably between about 0.45 and 0.55 moles.

A further aspect of the present invention is a process for preparing said approximately hemihydrate crystalline form of ziprasidone hydrochloride, comprising the preparation of a solution or suspension of ziprasidone free base in an organic solvent in the presence of water and the subsequent precipitation of said approximately hemihydrate crystalline form of ziprasidone hydrochloride by the addition of hydrochloric acid. Ziprasidone free base can be dissolved or suspended in the organic solvent in the presence of water; or it can be prepared directly in situ, for example by synthesising it in the same solvent according to one of the known preparation methods. Preferably ziprasidone free base is dissolved or suspended in a suitable organic solvent or in a mixture of organic solvents in the presence of water before adding the hydrochloric acid. Examples of organic solvent are ethers, such as diisopropyl ether, diethyl ether, tetrahydrofuran, dioxane, methyl t-butyl ether. Examples of preferred solvents are tetrahydrofuran and dioxane.

The percentage of water with respect to organic solvent is between about 0.05 and 95% v/v, preferably between about 0.1 and 5% v/v. The concentration of ziprasidone free base in the starting solution can be between about 1 and 70% w/w, preferably between 5 and 40%. From the solution or from the resultant suspension the approximately hemihydrate ziprasidone hydrochloride can be obtained by adding hydrochloric acid in gaseous form or as a solution in water or an organic solvent. After cooling the solution or the resultant suspension, the approximately hemihydrate ziprasidone hydrochloride of the present invention is recovered by filtering, washing with the same solvent as used in the reaction and drying under vacuum until constant weight is achieved. The drying temperature of the product obtained depends on the solvent used in the process and is between 0° C. and the boiling point of the solvent used, preferably between 20 and 70° C.

Approximately hemihydrate ziprasidone hydrochloride can be used for the treatment of pathologies in which ziprasidone hydrochloride is used, for example as described in U.S. Pat. No. 4,831,031.

The invention also provides a pharmaceutical composition comprising ziprasidone hydrochloride in an approximately hemihydrate crystalline form, optionally at least one of ziprasidone hydrochloride, ziprasidone hydrochloride monohydrate, ziprasidone hydrochloride hemihydrate and ziprasidone hydrochloride anhydrate as active principle, and at least one excipient and/or carrier.

A pharmaceutical composition in accordance with the invention can be formulated with known methods in any pharmaceutical form known for administering to mammals, including humans.

The dosage of the active ingredient for instance in the capsules, tablets, sugar coated tablets or other pharmaceutical forms for unitary oral administration may range from about 15 to about 80 mg.

A further aspect of the present invention comprises a method for purifying known crystalline forms of ziprasidone hydrochloride monohydrate, hemihydrate and anhydrate, comprising converting said forms into the new approximately hemihydrate crystalline form and reconverting this latter into ziprasidone hydrochloride monohydrate, hemihydrate and anhydrate, respectively.

In accordance with this process, the known ziprasidone hydrochloride monohydrate, hemihydrate and anhydrate are used as the starting materials for preparing a solution or a suspension of ziprasidone hydrochloride free base from which the new approximately hemihydrate crystalline form is obtained. A solution of this latter is in turn the starting material for obtaining the known ziprasidone hydrochloride monohydrate, hemihydrate or anhydrate, in accordance with known methods, for example from U.S. Pat. No. 5,312,926. The process used for preparing the new approximately hemihydrate crystalline form in fact enables the product to be purified from the impurities formed during the course of the synthesis process due to parasitic reactions and degradation of the product itself. Ziprasidone hydrochloride monohydrate, hemihydrate and anhydrate are therefore obtained at good yields and with a high degree of purity, being at least higher than 99.5%, thus satisfying the usual requirement established by the regulations governing the preparation of galenic formulations.

Interest in the new crystalline form therefore appears at the moment to lie, for example but not limited thereto, in a useful application in pharmaceutical technology.

The following non limiting example illustrates the invention.

### EXAMPLE

### Preparation of the Approximately Hemihydrate Form of Ziprasidone Hydrochloride

3 g ziprasidone free base (7.3 mmoles; water content according to Karl-Fischer method: 1.21%) and 50 ml of tetrahydrofuran are introduced into a 100 ml flask, equipped with a magnetic stirrer and dropping funnel and the suspension is heated to a temperature of 35° C. Then gaseous HCI, obtained by adding 96% H₂SO₄ into 37% HCI drop-wise, are bubbled into the suspension for 15 minutes. At the end of the acid addition, the suspension is left to cool to ambient temperature and filtered through a Buckner, finally drying the product under vacuum in an oven at a temperature of 50° C. 2.8 g of product are obtained (yield: 84.7%). M.p. :>290° C. (dec.) Chloride content (by potentiometric titration): 7.35%. Water content (by Karl-Fischer method):2.07%.

1. Approximately hemihydrate crystalline form of ziprasidone hydrochloride characterised by having an BRD spectrum wherein the most intense diffraction peaks are found at 7.44; 12.60; 13.89; 17.88; 20.94 and 25.98 in 2θ.

2. Crystalline form as claimed in claim 1 having an XPRD spectrum as shown in FIG. 4 of the accompanying drawings.

3. Crystalline form as claimed in claim 1 wherein the water content is within the range 0.4-0.6 moles per mole of ziprasidone hydrochloride.

4. Process for preparing the crystalline form of ziprasidone hydrochloride as claimed in claim 1, comprising the preparation of a solution or suspension of ziprasidone free base in an organic solvent in the presence of water and the subsequent precipitation of said approximately hemihydrate crystalline form by the addition of hydrochloric acid.

5. Process as claimed in claim 4, wherein the quantity of water compared to said organic solvent is between 0.05% and 95% v/v.

6. Process as claimed in claim 4, wherein The concentration of ziprasidone free base in the starting solution or suspension is between 1 and 70% w/w.

7. Pharmaceutical composition comprising as active principle ziprasidone hydrochloride in an approximately hemihydrate crystalline form as claimed in claim 1, and optionally at least one of ziprasidone hydrochloride, ziprasidone hydrochloride monohydrate, hemihydrate or anhydrate, and at least one excipient and/or carrier.

8. Purification method of a crystalline form of ziprasidone hydrochloride monohydrate, hemihydrate or anhydrate, comprising converting said form into the approximately hemihydrate crystalline form as claimed in claim 1, and reconverting this latter into ziprasidone hydrochloride monohydrate, hemihydrate or anhydrate, respectively.

9. Purification method of a crystalline form of ziprasidone hydrochloride monohydrate, hemihydrate or anhydrate, comprising converting said form into the approximately hemihydrate crystalline form as claimed in claim 2, and reconverting this latter into ziprasidone hydrochloride monohydrate, hemihydrate or anhydrate, respectively.
